(19) United States
(12) Patent Application Publication
(10) Pub. No.: US 2025/0236789 A1
(43) Pub. Date: Jul. 24, 2025

(54) SECOND NEAR-INFRARED ORGANIC FLUORESCENT PROBE, PREPARATION METHOD AND USE THEREOF

(71) Applicant: INNER MONGOLIA UNIVERSITY, Huhhot (CN)

(72) Inventors: Jianguo WANG, Huhhot (CN); Guoyu JIANG, Huhhot (CN); Chunbin LI, Huhhot (CN)

(21) Appl. No.: 18/741,551

(22) Filed: Jun. 12, 2024

(30) Foreign Application Priority Data
Jan. 23, 2024 (CN) 202410088933.4
Feb. 2, 2024 (WO) PCT/CN2024/075367

(51) Int. Cl. C09K 11/06; A61K 49/00

(52) U.S. Cl. CPC C09K 11/06; A61K 49/0021; C09K 2211/1051

(57) ABSTRACT

Provided are a second near infrared organic fluorescent probe, a preparation method and use thereof. The fluorescent probes is mixed with an organic coating agent to prepare a nanoimaging agent. The nanoimaging agents have a high molar extinction coefficient and a high quantum yield, and exhibit bright NIR-II fluorescence under excitation of white light. Furthermore, the nanoimaging agents could achieve high-resolution imaging of blood vessels under excitation of white light. In addition, using white light as excitation light source in the imaging process could effectively avoid problems caused by single wavelength excitation light source, such as limited photon absorption, laser-induced biological damage, and uneven irradiation. Moreover, laser light source is high in cost, while white light source is low in cost and easily available. The second near infrared organic fluorescent probe may be synthesized by a one-step Knoevenagel reaction without any metal catalysis.

### CROSS-REFERENCE TO RELATED APPLICATION

This application claims priority to International Patent Application No. PCT/CN2024/075367, filed on Feb. 2, 2024, which claims priority of the Chinese Patent Application No. 202410088933.4, filed with the China National Intellectual Property Administration (CNIPA) on Jan. 23, 2024. The disclosure of the two patent applications is incorporated by references in their entireties.

### TECHNICAL FIELD

The present disclosure belongs to the technical field of biochemical materials, and specifically relates to a second near-infrared organic fluorescent probe, a preparation method and use thereof.

### BACKGROUND

In vivo imaging technology refers to a technology for qualitative and quantitative study on biological processes in vivo at the tissue, cellular, and molecular levels without damaging animals using imaging methods. This technology allows for non-invasive and intuitive observation of various biological processes in living animals and has received widespread attention for advantages such as high sensitivity, high spatiotemporal resolution, and real-time monitoring ability, which is of great significance for fields such as life sciences and medicine. As the core of in vivo fluorescence imaging technology, high-performance fluorescence imaging agents have always been a research focus for researchers. Second near-infrared (NIR-II) organic fluorescent molecules have become an ideal choice for high-performance fluorescence imaging agents due to their greater tissue penetration depth and low spontaneous fluorescence interference. However, NIR-II organic fluorescent molecules have relatively long absorption and emission wavelengths, a relatively small band gap, and an excited state energy that is easily attenuated by non-radiative transition, and thus have a relatively low luminous efficiency. Therefore, it is of great significance to develop NIR-II organic nanoimaging agents with high luminous efficiency.

In another aspect, when using NIR-II organic nanoimaging agents for in vivo fluorescence imaging, expensive lasers with specific wavelength(s) are needed to be used as excitation light sources, because of the long absorption wavelength, low quantum yield, and limited molar extinction coefficient of NIR-II organic fluorescence molecules. Furthermore, the use of single-wavelength lasers also brings about problems such as limited photon absorption and laser-induced biological damage. In addition, single-wavelength lasers could not provide uniform irradiation, and the energy provided thereby would decay from the center of the spot to the surrounding, which would also adversely affect the imaging quality of in vivo imaging. In contrast, white light, including ordinary lamps, surgical shadowless lamps, and laparoscopic light sources, as a safe and visible excitation light source, has the characteristics of a wide continuous spectrum, low cost, and easy availability, and is an ideal choice for excitation light sources in in vivo fluorescence imaging. However, use of white light as an excitation light source in NIR-II fluorescence imaging has not been reported yet, which requires NIR-II organic nanoimaging agents to have high quantum yields and molar extinction coefficients. Therefore, the development of high-performance NIR-II organic nanoimaging agents excited by white light is urgently needed, but faces great challenges.

### SUMMARY

An object of the present disclosure is to provide a preparation method and use of A-D-A type second near-infrared (NIR-II) organic fluorescent probe excited by white light. The fluorescent probe according to the present disclosure and an organic coating agent is mixed to prepare a nanoimaging agent, which has good biocompatibility and biological stability, a high molar extinction coefficient and a high quantum yield, and bright second near-infrared fluorescence under excitation of white light, and thus could be used to construct a high-performance bioimaging contrast agent and has broad prospects in the fields such as non-therapeutic in vivo imaging, and image-guided surgery.

In order to achieve the above objects, the present disclosure provides the following technical solutions:

The present disclosure provides a second near infrared (NIR-II) organic fluorescent probe having a structure represented by formula I:

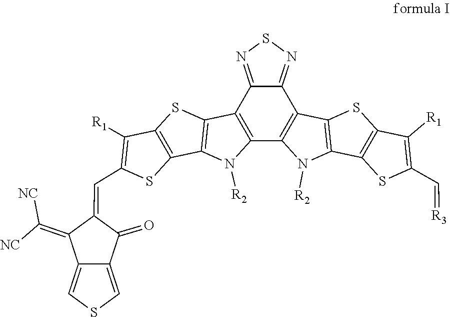

where in formula I,
R₁ and R₂ each are independently C₁₋₁₆ branched or linear alkyl;
R₃ is one selected from the group consisting of

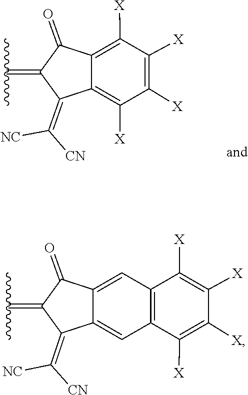

X in R₃ being at least one selected from the group consisting of H and halogen.

In some embodiments, R₁ and R₂ each are independently C₁₋₁₁ branched or linear alkyl.

In some embodiments, X in R₃ is at least one selected from the group consisting of H, F, Cl and Br.

In some embodiments, the NIR-II organic fluorescent probe has a structure represented by formula I-a, I-b or formula I-c;

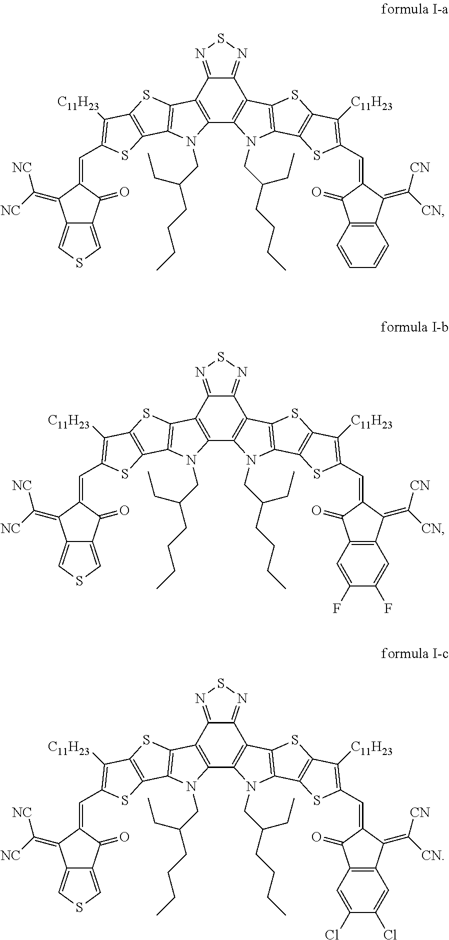

The present disclosure provides a method for preparing the NIR-II organic fluorescent probe of the above technical solutions, comprising the following steps:
mixing a compound having a structure represented by formula II, a compound having a structure represented by formula III, a compound having a structure represented by formula IV or formula V, an organic base catalyst and an organic solvent, and performing a Knoevenagel reaction to obtain the NIR-II organic fluorescent probe having the structure represented by formula I;

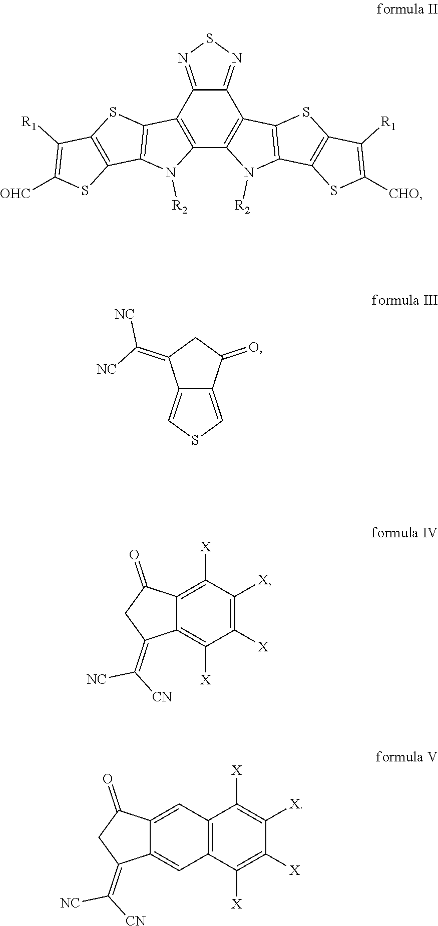

In some embodiments, a molar ratio of the compound having the structure represented by formula II, the compound having the structure represented by formula III, and the compound having the structure represented by formula IV or formula V is 1:1:1,
the organic base catalyst is pyridine, and a molar ratio of the compound having the structure represented by formula II to the organic base catalyst is in a range of 1:3 to 1:10, and
the Knoevenagel reaction is performed at a temperature of 25° C. to 70° C. for 8 h to 24 h.

The present disclosure provides a fluorescent probe coated nanoparticle, comprising an NIR-II organic fluorescent probe of the above technical solutions or an NIR-II organic fluorescent probe prepared by the method of the above technical solutions, and an organic coating agent coating on a surface of the NIR-II organic fluorescent probe.

The present disclosure provides a method for preparing the fluorescent probe coated nanoparticle of the above technical solutions, comprising the following steps:
mixing the NIR-II organic fluorescent probe of the above technical solutions or the NIR-II organic fluorescent probe prepared by the method of the above technical solutions, an organic coating agent and an organic solvent to obtain a mixed solution; and
mixing the mixed solution with water, and performing ultrasonic coprecipitation to obtain the fluorescent probe coated nanoparticle.

The present disclosure provides use of the NIR-II organic fluorescent probe of the above technical solutions or the NIR-II organic fluorescent probe prepared by the method of the above technical solutions or the fluorescent probe-coated nanoparticle of the above technical solutions or the fluorescent probe-coated nanoparticle prepared by the method of the above technical solutions in preparation of a bioimaging contrast agent.

The present disclosure provides use of the NIR-II organic fluorescent probe of the above technical solutions or the NIR-II organic fluorescent probe prepared by the method of the above technical solutions or the fluorescent probe-coated nanoparticle of the above technical solutions or the fluorescent probe-coated nanoparticle prepared by the method of the above technical solutions in fluorescence imaging for non-diagnostic purposes and non-therapeutic purposes.

The present disclosure provides a NIR-II organic fluorescent probe, having a structure represented by formula I. The fluorescent probe according to the present disclosure has an A-D-A (receptor-donor-receptor structure) type large π conjugate plane structure. The fluorescent probe according to the present disclosure is mixed with an organic coating agent to prepare nanoparticles, which could be applied to imaging agents and has good biocompatibility and biological stability. The fluorescent probe and the nanoparticles as nanoimaging agents according to the present disclosure have a high molar extinction coefficient and a high quantum yield, and exhibit bright NIR-II fluorescence under excitation of white light. Furthermore, the fluorescent probe according to the present disclosure could achieve high-resolution imaging of blood vessels under excitation of white light. In addition, using white light as excitation light source in the imaging process could effectively avoid problems caused by single wavelength excitation light source, such as limited photon absorption, laser-induced biological damage, and uneven irradiation. Therefore, the fluorescent probe having the structure represented by formula I according to the present disclosure could be used as a nanoimaging agent to construct a high-performance bioimaging contrast agent, and has broad prospects in the fields such as non-therapeutic in vivo imaging, image-guided surgery.

The results of examples show that the fluorescent probe molecule having the structure represented by formula I according to the present disclosure is a receptor-donor-receptor structure, which could effectively prolong the absorption and emission windows and even the near infrared window, and has the characteristics of a high molar extinction coefficient and a wide absorption band, thus greatly improving the white light absorption capacity of the fluorescent probe. The nanoimaging agent according to the present disclosure emits bright NIR-II fluorescence under excitation of white light, and its emission wavelength could be extended to 1400 nm, which could effectively avoid self-absorption and reduce background interference.

The present disclosure provides a method for preparing an NIR-II organic fluorescent probe of the above technical solutions. The method according to the present disclosure is simple in synthesis, which could result in the NIR-II organic fluorescent probe only by a one-step Knoevenagel reaction without any metal catalysis, easy to operate, and suitable for industrial production.

### BRIEF DESCRIPTION OF THE DRAWINGS

FIGS. 1A-1D show particle sizes of nanoimaging agents HY6CT-NPs, FY6CT-NPs and CY6CT-NPs and a transmission electron microscope (TEM) image of the nanoimaging agent HY6CT-NPs, in which FIG. 1A shows a TEM image of the nanoimaging agent HY6CT-NPs; FIGS. 1B-1D show particle sizes of the nanoimaging agents HY6CT-NPs, FY6CT-NPs and CY6CT-NPs, respectively.

FIG. 2 shows absorption spectra of nanoimaging agents HY6CT-NPs, FY6CT-NPs and CY6CT-NPs in aqueous solution.

FIG. 3 shows emission spectra of aqueous solutions of nanoimaging agents HY6CT-NPs, FY6CT-NPs and CY6CT-NPs under excitation of white light.

FIG. 4 shows test results of relative quantum yields of nanoimaging agents HY6CT-NPs, FY6CT-NPs and CY6CT-NPs with a commercial dye IR26 as reference.

FIG. 5 shows NIR-II imagings of aqueous solutions of HY6CT-NPs, FY6CT-NPs, CY6CT-NPs and ICG with the same concentration under excitation of white light.

FIGS. 6A-6D show NIR-II fluorescence imaging of the nanoimaging agent FY6CT-NPs on the abdominal blood vessels in a normal mouse under different filter conditions and resolution analysis graphs under different filter conditions, in which FIG. 6A shows the NIR-II fluorescence imaging of the abdominal blood vessels in a normal mouse with the nanoimaging agent Y6CT-NPs under different filter conditions; and FIGS. 6B-6D show resolution analysis graphs under different filter conditions.

### DETAILED DESCRIPTION OF THE EMBODIMENTS

The present disclosure provides a second near infrared (NIR-II) organic fluorescent probe, having a structure represented by formula I.

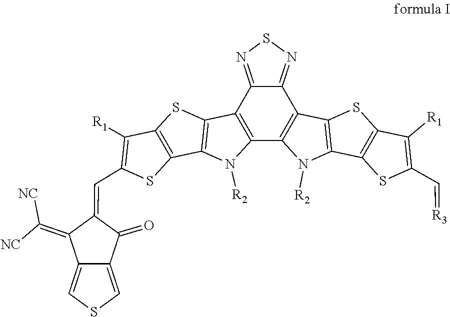

where in formula I,
R₁ and R₂ each are independently C₁₋₁₆ branched or linear alkyl, and
R₃ is one selected from the group consisting of

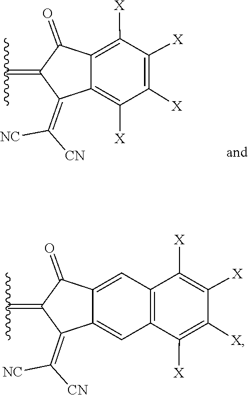

X in R₃ being at least one selected from the group consisting of H and halogen.

In some embodiments of the present disclosure, R₁ is C₁₋₁₁ branched or linear alkyl, preferably C₃₋₁₁ branched or linear alkyl, and more preferably C₅₋₁₁ branched or linear alkyl.

In a specific embodiment of the present disclosure, R₁ is C₁₁H₂₃ linear alkyl.

In some embodiments of the present disclosure, R₂ is C₁₋₁₁ branched or linear alkyl, preferably C₃₋₁₁ branched or linear alkyl, and more preferably C₅₋₈ branched or linear alkyl.

In a specific embodiment of the present disclosure, R₂ is

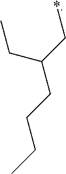

In some embodiments of the present disclosure, R₃ is

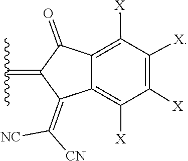

In some embodiments of the present disclosure, X in R₃ is at least one selected from the group consisting of H, F, Cl and Br, and preferably at least one selected from the group consisting of H, F and Cl.

In a specific embodiment of the present disclosure, X in R₃ is H, or H and F, or H and Cl.

In some embodiments of the present disclosure, the NIR-II organic fluorescent probe having the structure represented by formula I has a structure represented by formula I-a, formula I-b or formula I-c,

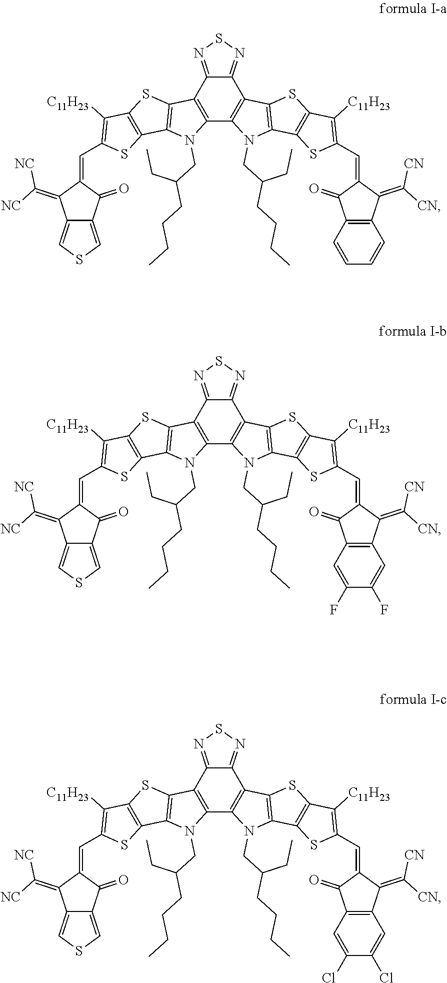

where R₁ in formula I-a, formula I-b or formula I-c is C₁₁H₂₃ linear alkyl.

The present disclosure provides a method for preparing a NIR-II organic fluorescent probe of the above technical solutions, comprising the following steps:
mixing a compound having a structure represented by formula II, a compound having a structure represented by formula III, a compound having a structure represented by formula IV or formula V, an organic base catalyst and an organic solvent, and performing a Knoevenagel reaction to obtain the NIR-II organic fluorescent probe having the structure represented by formula I;

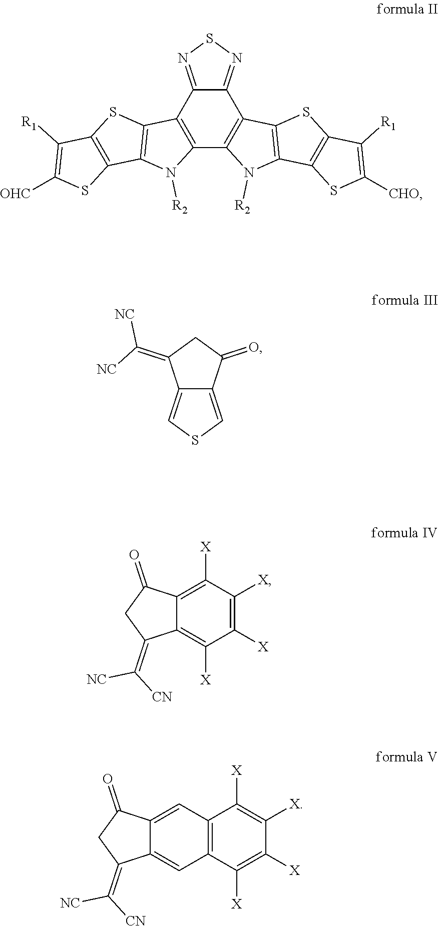

In the present disclosure, unless otherwise specified, all preparation raw materials/components are commercially available products well known to those skilled in the art.

In some embodiments of the present disclosure, the compound having the structure represented by formula IV is a dicyanoindanone compound having a structure represented by formula IV-a, formula IV-b or formula IV-c,

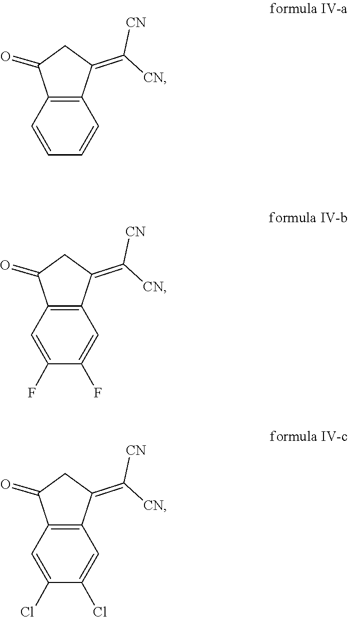 [SMILES: N#CC(C#N)=C1CC(=O)c2ccccc21 ; N#CC(C#N)=C1CC(=O)c2cc(F)c(F)cc21 ; N#CC(C#N)=C1CC(=O)c2cc(Cl)c(Cl)cc21]

In some embodiments of the present disclosure, a molar ratio of the compound having the structure represented by formula II, the compound having the structure represented by formula III, and the compound having the structure represented by formula IV or formula V is 1:1:1.

In some embodiments of the present disclosure, the organic base catalyst is pyridine. In some embodiments of the present disclosure, a molar ratio of the compound having the structure represented by formula II to the organic base catalyst is in a range of 1:3 to 1:10, preferably 1:5.

In some embodiments of the present disclosure, the organic solvent is chloroform. In the present disclosure, there is no special limitation on the amount of the organic solvent, as long as the reaction could proceed smoothly. In the present disclosure, there is no special requirement for the specific operation of the mixing.

In some embodiments of the present disclosure, the Knoevenagel reaction is performed at a temperature of 25-70° C., preferably 40-60° C. In some embodiments of the present disclosure, the Knoevenagel reaction is performed for 8-24 h, preferably 12-24 h.

In some embodiments of the present disclosure, the Knoevenagel reaction is performed under a protective gas, and in further embodiments of the present disclosure, the protective gas is nitrogen or argon. In some embodiments of the present disclosure, the reaction is monitored by a TLC plate (i.e., thin layer chromatography spot plate).

In some embodiments of the present disclosure, the method further comprises after the Knoevenagel reaction, subjecting a resulting Knoevenagel reaction solution to post-treatment, the post-treatment comprising the following steps:
subjecting the resulting Knoevenagel reaction solution to extraction and concentration to obtain a concentrated solution, and
subjecting the concentrated solution to column chromatography separation and recrystallization to obtain a pure product of the NIR-II organic fluorescent probe having the structure represented by formula I.

In some embodiments of the present disclosure, the extraction comprises the following steps: after the Knoevenagel reaction solution is cooled to ambient temperature, water is added into the Knoevenagel reaction solution, and an aqueous phase is extracted a plurality of times with dichloromethane. In some embodiments of the present disclosure, a resulting organic phase from extraction is concentrated. In the present disclosure, there is no special requirement for the concentration method, and a concentration method well known to those skilled in the art may be used.

In some embodiments of the present disclosure, an eluent for the column chromatography separation is a mixed solution of dichloromethane and petroleum ether, wherein a volume ratio of the dichloromethane to the petroleum ether is 2:1. In some embodiments of the present disclosure, after the column chromatography separation is completed, a solvent in a column chromatography product is removed. In the present disclosure, there is no special limitation on the method for removing the solvent, and a conventional method for removing the solvent, such as rotary steaming, may be used.

In some embodiments of the present disclosure, a solvent for the recrystallization is a mixture of dichloromethane and n-hexane. A volume ratio of the dichloromethane to the n-hexane is in a range of 1:3 to 1:6. A specific embodiment of the recrystallization comprises: dissolving a crude product from the column chromatography separation in dichloromethane, slowly adding n-hexane under ultrasonic condition, and filtering after a target product is precipitated, and obtaining a solid, i.e., the target product.

The present disclosure provides a fluorescent probe coated nanoparticle, comprising an NIR-II organic fluorescent probe of the above technical solutions or an NIR-II organic fluorescent probe prepared by the method of the above technical solutions, and an organic coating agent coating on a surface of the NIR-II organic fluorescent probe.

In some embodiments of the present disclosure, the organic coating agent is at least one selected from the group consisting of methoxypolyethylene glycol amine, 1,2-distearoyl-sn-glycero-3-phosphoethanolamine-poly(ethylene glycol) (DSPE-PEG), 1,2-distearoyl-sn-glycero-3-phosphoethanolamine-N-[maleimide(polyethylene glycol)], 1,2-distearoyl-sn-glycero-3-phosphoethanolamine-N-[folate(polyethylene glycol)], 1,2-distearoyl-sn-glycero-3-phosphoethanolamine-N-[thiol(polyethylene glycol)], 1,2-distearoyl-sn-glycero-3-phosphoethanolamine-N-[carboxy(polyethylene glycol)], 1,2-distearoyl-sn-glycero-3-phosphoethanolamine-N-[azido(polyethylene glycol)], 1,2-distearoyl-sn-glycero-3-ethanolamine-N-[biotin(polyethylene glycol)], 1-palmitoyl-2-oleoyl-sn-glycero-3-phosphoethanolamine, 1-stearoyl-2-oleoyl lecithin, 1,2-dipalmitoyl phosphoethanolamine-polyethylene glycol, polystyrene-grafted-poly(ethylene glycol), methoxy PEG polylactic acid-hydroxyacetic acid copolymer, and Pluronic® F-127.

In some embodiments of the present disclosure, the fluorescent probe coated nanoparticle has a particle size of 50 nm to 200 nm, preferably 100 nm to 200 nm.

The present disclosure provides a method for preparing the fluorescent probe coated nanoparticle of the above technical solutions, comprising the following steps:
mixing the NIR-II organic fluorescent probe of the above technical solutions or the NIR-II organic fluorescent probe prepared by the method of the above technical solutions, an organic coating agent and an organic solvent to obtain a mixed solution, and
mixing the mixed solution with water, and performing ultrasonic coprecipitation to obtain the fluorescent probe coated nanoparticle.

In the present disclosure, the NIR-II organic fluorescent probe of the above technical solutions or the NIR-II organic fluorescent probe prepared by the method of the above technical solutions, an organic coating agent and an organic solvent are mixed to obtain a mixed solution. In some embodiments of the present disclosure, a mass ratio of the fluorescent probe to the organic coating agent is in a range of 1:3 to 1:8, preferably 1:5 to 1:6. In some embodiments of the present disclosure, the organic solvent is tetrahydrofuran. In the present disclosure, there is no special requirement for the amount of the organic solvent.

After the mixed solution is obtained, the mixed solution is mixed with water, and ultrasonic coprecipitation is performed to obtain the fluorescent probe coated nanoparticle. In some embodiments of the present disclosure, the ultrasonic coprecipitation is performed at a powder of 100 W to 200 W, preferably 150 W; and the ultrasonic coprecipitation is performed for 3 min to 10 min, preferably 5 min.

In some embodiments of the present disclosure, an assembly liquid is directly obtained after the ultrasonic coprecipitation, and the method of the present disclosure further comprises: introducing the assembly liquid into a dialysis bag, and performing dialysis to obtain a purified assembly material; and concentrating the purified assembly material to obtain a solution of the nanoimaging agent. In some embodiments of the present disclosure, the dialysis bag has a molecular weight cutoff of 3500; and the dialysis is performed for 48 h to 72 h. In some embodiments of the present disclosure, the concentration is performed by water absorption using polyethylene glycol, and the polyethylene glycol has an average molecular weight of 100,000. A concentrated solution is obtained by the concentration. In some embodiments of the present disclosure, a syringe filter is used to filter the concentrated solution to remove impurities so as to obtain the solution of the nanoimaging agent.

In the present disclosure, the fluorescent probe is prepared into nanoparticles dispersed in water, which is beneficial to imaging blood vessels for non-therapeutic purpose and non-diagnostic purposes under excitation of white light.

The present disclosure provides use of the NIR-II organic fluorescent probe of the above technical solutions or the NIR-II organic fluorescent probe prepared by the method of the above technical solutions or the fluorescent probe coated nanoparticle of the above technical solutions or the fluorescent probe coated nanoparticle prepared by the method of the above technical solutions in preparation of a bioimaging contrast agent.

In some embodiments of the present disclosure, the bioimaging contrast agent is used for monitoring blood vessel imaging in a liver ischemia-reperfusion process, or monitoring blood vessel imaging in a kidney transplantation process, or for blood vessel imaging in a fluorescent image-guided surgery.

The present disclosure provides use of the NIR-II organic fluorescent probe of the above technical solutions or the NIR-II organic fluorescent probe prepared by the method of the above technical solutions or the fluorescent probe-coated nanoparticle of the above technical solutions or the fluorescent probe-coated nanoparticle prepared by the method of the above technical solutions in fluorescence imaging for non-diagnostic purposes and non-therapeutic purposes.

In some embodiments of the present disclosure, the fluorescence imaging for non-diagnostic purposes and non-therapeutic purposes is vascular fluorescence imaging.

In some embodiments of the present disclosure, the fluorescence imaging for non-diagnostic purposes and non-therapeutic purposes is in vivo imaging.

In specific embodiments of the present disclosure, the use in fluorescence imaging comprises monitoring the vascular fluorescence imaging in a liver ischemia-reperfusion process, or monitoring the vascular fluorescence imaging in a kidney transplantation process, or vascular fluorescence imaging in a fluorescent image-guided surgery.

In some embodiments of the present disclosure, a device for the blood vessel imaging is a NIR-II in vivo imaging instrument.

In some embodiments of the present disclosure, the fluorescence imaging is performed under excitation of white light, and an excitation light source of the white light is an NIR-II in vivo imaging instrument illuminating lamp, with a wavelength being 400 nm to 800 nm.

In some embodiments of the present disclosure, a nanoimaging agent injected into a mouse body during the vascular fluorescence imaging has an effective concentration of not less than 500 mol L⁻¹, and a volume of not less than 100 μL.

In order to further illustrate the present disclosure, the technical solutions of the present disclosure will be described in detail in conjunction with examples below, but these examples cannot be understood as limiting the scope of the present disclosure.

### Example 1

This example was performed according to the following reaction flow:

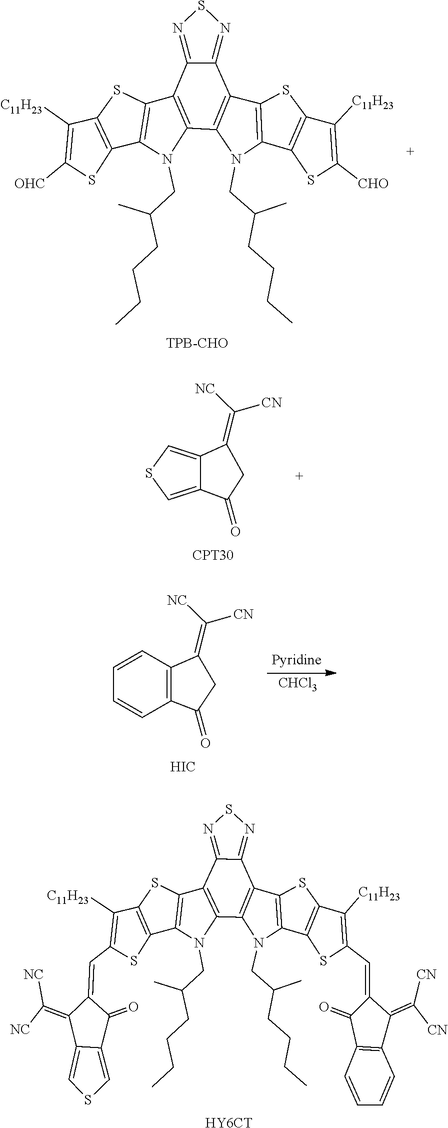

TPB-CHO (103 mg, 0.1 mmol), CPT30 (20 mg, 0.1 mmol) and IC (20 mg, 0.1 mmol) were dissolved in 10 mL of chloroform. Pyridine (40 mg, 0.5 mmol) was added dropwise into a resulting solution to obtain a mixture, and the mixture was reacted at 50° C. for 12 h. A resulting reaction solution was cooled to ambient temperature, and then evaporated in vacuum to remove solvent to obtain a residue. The residue was dissolved in dichloromethane (30 mL) and extracted with water (30 mL). Resulting organic phases were combined, dried with anhydrous Na₂SO₄, and purified by silica gel column chromatography using petroleum ether/dichloromethane 2:1 as an eluent. A crude product was dissolved in dichloromethane, recrystallized by slowly adding n-hexane under ultrasonic condition, and after a solid was precipitated, a resulting solution was filtered to obtain a brown-black powder HY6CT (yield: 78 mg, 55%).

¹H NMR (600 MHz, Chloroform-d) δ 9.11 (s, 1H), 9.00 (s, 1H), 8.66 (d, J=7.0 Hz, 1H), 8.33 (s, 1H), 7.98-7.96 (m, 2H), 7.78-7.74 (m, 2H), 4.80 (t, J=8.7 Hz, 4H), 3.35-3.10 (m, 4H), 2.21-2.17 (m, 2H), 1.87-1.84 (m, 4H), 1.51-1.47 (m, 4H), 1.38-0.96 (m, 44H), 0.86-0.80 (m, 12H), 0.71-0.65 (m, 6H).

¹³C NMR (151 MHz, Chloroform-d) δ 188.53, 181.68, 156.66, 153.23, 153.01, 147.32, 145.03, 144.93, 142.60, 142.34, 140.04, 137.84, 136.89, 136.17, 135.29, 135.04, 134.28, 133.60, 133.32, 133.25, 127.32, 126.98, 125.18, 123.63, 120.86, 115.35, 113.63, 113.50, 68.28, 55.67, 40.38, 40.32, 31.91, 31.14, 29.82, 29.64, 29.62, 29.52, 29.50, 29.33, 27.63, 27.55, 23.29, 22.89, 22.84, 22.67, 14.10, 13.75, 13.71, 10.38, 10.34.

### Example 2

This example was performed according to the following reaction flow:

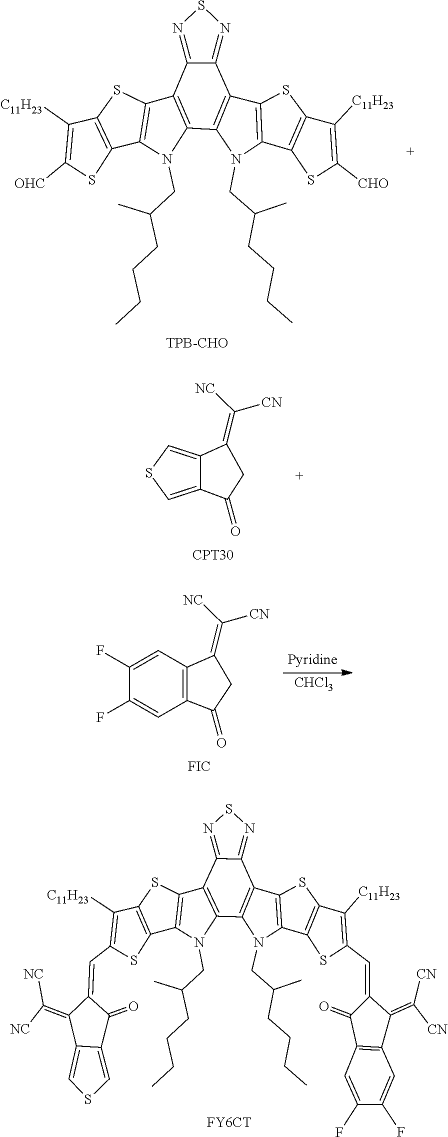

TPB-CHO (103 mg, 0.1 mmol), CPT30 (20 mg, 0.1 mmol) and FIC (23 mg, 0.1 mmol) were dissolved in 10 mL of chloroform. Pyridine (40 mg, 0.5 mmol) was added dropwise into a resulting solution to obtain a mixture, and the mixture was reacted at 50° C. for 12 h. A resulting reaction solution was cooled to ambient temperature, and then evaporated in vacuum to remove solvent to obtain a residue, and the residue was dissolved in dichloromethane (30 mL) and extracted with water (30 mL). Resulting organic phases were combined, dried with anhydrous Na₂SO₄, and purified by silica gel column chromatography using petroleum ether/dichloromethane 2:1 as an eluent. A crude product was dissolved in dichloromethane, recrystallized by slowly adding n-hexane under ultrasonic condition, and after a solid was precipitated, a resulting solution was filtered to obtain a brown-black powder FY6CT (yield: 82 mg, 58%).

¹H NMR (600 MHz, Chloroform-d) δ 9.16 (s, 1H), 9.09 (s, 1H), 8.57 (s, 1H), 8.40 (s, 1H), 7.97 (s, 1H), 7.71 (t, J=7.2, 1H), 4.79-4.75 (m, 4H), 3.24-3.20 (m, 4H), 2.17-2.09 (m, 2H), 1.88-1.84 (m, 4H), 1.52-1.48 (m, 4H), 1.37-0.96 (m, 44H), 0.87-0.74 (m, 12H), 0.67-0.64 (m, 6H).

¹³C NMR (151 MHz, Chloroform-d) δ 186.09, 181.60, 158.48, 153.84, 153.09, 147.24, 145.12, 138.00, 137.78, 136.05, 136.01, 135.74, 133.34, 133.28, 133.16, 130.33, 129.79, 127.02, 125.29, 119.72, 114.96, 114.51, 113.77, 113.57, 66.49, 55.74, 40.42, 40.35, 31.90, 31.18, 31.10, 29.81, 29.71, 29.63, 29.61, 29.51, 29.46, 29.32, 27.58, 23.35, 23.27, 22.93, 22.85, 22.67, 14.09, 13.78, 13.71, 13.68, 10.44, 10.36.

### Example 3

This example was performed according to the following reaction flow:

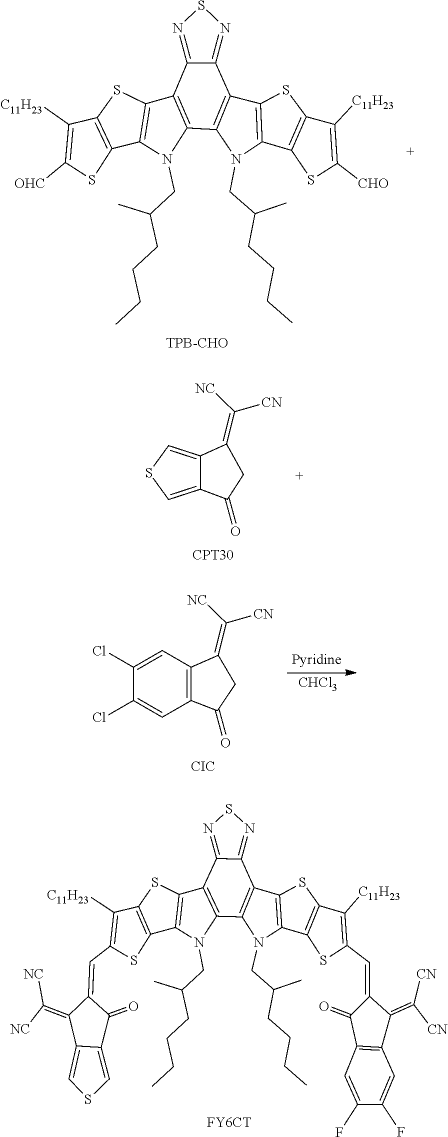

TPB-CHO (103 mg, 0.1 mmol), CPT30 (20 mg, 0.1 mmol) and CIC (26 mg, 0.1 mmol) were dissolved in 10 mL of chloroform. Pyridine (40 mg, 0.5 mmol) was added dropwise to a resulting solution to obtain a mixture, and the mixture was reacted at 50° C. for 12 h. A resulting reaction solution was cooled to ambient temperature, and then evaporated in vacuum to remove solvent to obtain a residue, and the residue was dissolved in dichloromethane (30 mL) and extracted with water (30 mL). Resulting organic phases were combined, dried with anhydrous Na₂SO₄, and purified by silica gel column chromatography using petroleum ether/dichloromethane 2:1 as an eluent. A crude product was dissolved in dichloromethane, recrystallized by slowly adding n-hexane under ultrasonic condition, and after a solid was precipitated, a resulting solution was filtered to obtain a brown-black powder CY6CT (yield: 75 mg, 52%).

¹H NMR (600 MHz, Chloroform-d) δ 9.07 (s, 1H), 9.00 (s, 1H), 8.70 (s, 1H), 8.33 (s, 1H), 7.97 (d, J=12.1 Hz, 2H), 4.81 (t, J=10.6 Hz, 4H), 3.20-3.14 (m, 4H), 2.20-2.17 (m, 2H), 1.86-1.84 (m, 4H), 1.49-1.46 (m, 4H), 1.35-0.96 (m, 44H), 0.87-0.80 (m, 12H), 0.70-0.65 (m, 6H).

¹³C NMR (151 MHz, Chloroform-d) δ 186.12, 181.59, 153.12, 147.39, 145.16, 142.60, 139.47, 139.12, 138.72, 138.04, 136.41, 136.11, 135.53, 134.03, 133.50, 133.44, 133.35, 130.82, 129.98, 127.34, 127.17, 126.80, 125.20, 124.93, 119.73, 115.05, 114.96, 114.55, 113.54, 55.80, 40.46, 40.42, 31.89, 31.16, 31.08, 29.80, 29.71, 29.62, 29.59, 29.49, 29.46, 29.43, 29.30, 27.73, 27.68, 23.42, 23.34, 22.85, 22.81, 22.65, 14.05, 13.71, 13.67, 10.37, 10.31.

### Example 4

This example was performed according to the following reaction flow:

2 mg of fluorescent probes (HY6CT prepared according to Example 1, FY6CT prepared according to Example 2 and CY6CT prepared according to Example 3) and 10 mg of DSPE-PEG₂₀₀₀ were dissolved in 1 mL of tetrahydrofuran, and then added into 10 mL of deionized water and subjected to ultrasonic assembly. After that, a resulting nanoparticle solution was transferred into a dialysis bag with a molecular weight cutoff of 3,500, and dialyzed for purification for 72 hours. The dialyzed nanoparticle solution was concentrated by absorbing water using polyethylene glycol with an average molecular weight of 100,000, and then filtered by a syringe filter to remove impurities. Finally, a fluorescent probe coated nanoparticle (nanoimaging agent, named HY6CT-NPs, FY6CT-NPs and CY6CT-NPs respectively) was obtained. The effective concentration of the nanoimaging agent was determined to be 500 μmol·L⁻¹ by the pre-established concentration curve.

### Performance Test:

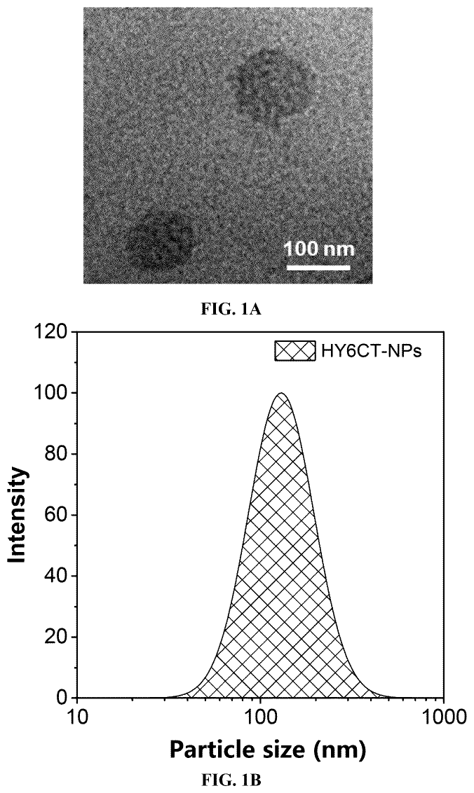

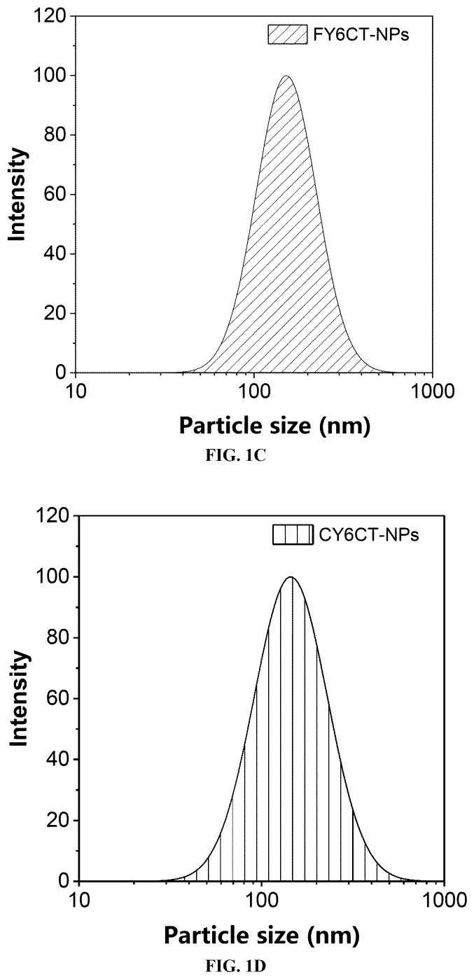

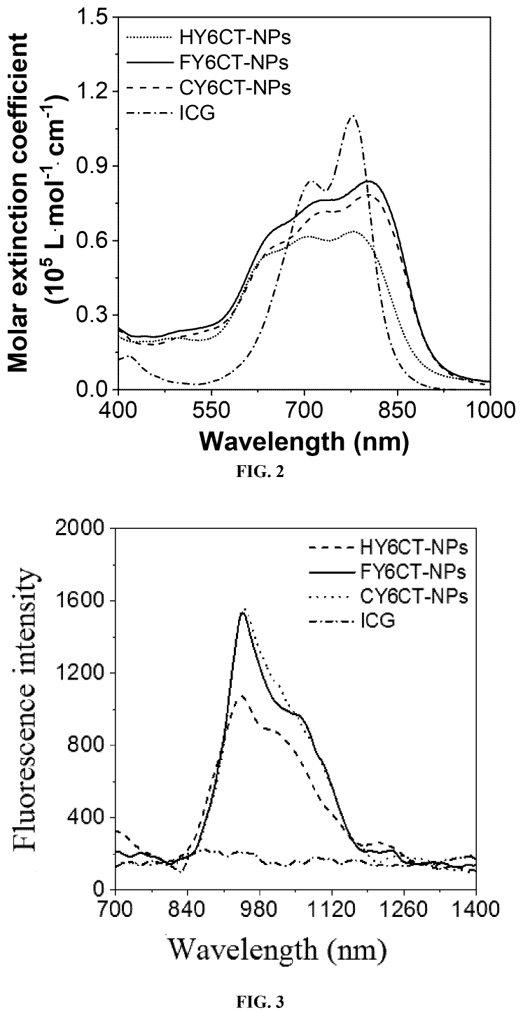

(1) Particle size test of the fluorescent probe coated nanoparticles HY6CT-NPs, FY6CT-NPs and CY6CT-NPs: 30 L of fluorescent nanoprobe was added into 3 mL of deionized water, and the particle size was then tested by a transmission electron microscope and a dynamic light scattering instrument, respectively. The results are shown in FIGS. 1A-1D.

FIG. 1A shows a transmission electron microscope image of the nano fluorescent probe HY6CT-NPs, and FIGS. 1B-1D show particle sizes of HY6CT-NPs, FY6CT-NPs and CY6CT-NPs, respectively. As can be seen from FIGS. 1A-1D, HY6CT-NPs, FY6CT-NPs and CY6CT-NPs have particle sizes of 130 nm, 149 nm and 141 nm, respectively.

(2) Absorption spectra test of nano fluorescent probes: The absorption spectra of HY6CT-NPs, FY6CT-NPs, CY6CT-NPs and ICG in aqueous solution were tested with an ultraviolet-visible spectrophotometer equipped with an integrating sphere module, at a test concentration of 10 μmol·L⁻¹. The results are shown in FIG. 2.

FIG. 2 shows absorption spectra of fluorescent probe coated nanoparticles HY6CT-NPs, FY6CT-NPs and CY6CT-NPs in aqueous solution. As can be seen from FIG. 2, the three fluorescent probe coated nanoparticles HY6CT-NPs, FY6CT-NPs and CY6CT-NPs all show high absorption rates in the range of 400-1000 nm, and HY6CT-NPs, FY6CT-NPs and CY6CT-NPs have molar extinction coefficients at the maximum absorption peak of 6.35×10⁴ L·mol⁻¹·cm⁻¹ (780 nm), 8.38×10⁴ L·mol⁻¹·cm⁻¹ (806 nm) and 7.85×10⁴ L·mol⁻¹·cm⁻¹ (803 nm), respectively. Compared with ICG, nano fluorescent probes show wider absorption in the white light region (400-800 nm).

(3) Emission spectrum test of fluorescent probe coated nanoparticles: The emission spectra of HY6CT-NPs, FY6CT-NPs and CY6CT-NPs in aqueous solution were tested with steady-state transient fluorescence spectrometer, at a test concentration of 10 μmol·L⁻¹. The results are shown in FIG. 3.

Notably, three kinds of fluorescent probe coated nanoparticles show fluorescence emission in NIR-II region which could extend to 1400 nm under excitation of white light, and the maximum emission peaks of HY6CT-NPs, FY6CT-NPs and CY6CT-NPs are 947 nm, 948 nm and 954 nm, respectively. These results indicate that HY6CT-NPs, FY6CT-NPs and CY6CT-NPs could emit fluorescence in NIR-II region under excitation of white light. On the contrary, the spectrum of ICG can hardly be detected at the same concentration.

(4) Relative quantum yield test of fluorescent probe coated nanoparticles HY6CT-NPs, FY6CT-NPs and CY6CT-NPs in aqueous solution: the NIR-II commercial fluorescent dye IR26 was selected as a reference to test the relative quantum yields of HY6CT-NPs, FY6CT-NPs and CY6CT-NPs. IR26 needs to be prepared into a solution in dichloroethane (DCE). Firstly, the absorption spectra of HY6CT-NPs, FY6CT-NPs, CY6CT-NPs and IR26-DCE were tested separately to determine their concentrations corresponding to absorbance values at 808 nm of 0.02, 0.04, 0.06, 0.08, and 0.10, respectively. The fluorescence spectra of the four at their corresponding concentrations were tested when excited at 808 nm. The fluorescence spectra with emission wavelengths ranging from 850 to 1,400 nm were integrated and used as the y-axis, and the absorbance values were used as the x-axis, and a linear regression analysis was performed to obtain the slope. The relative quantum yields of HY6CT-NPs, FY6CT-NPs and CY6CT-NPs were calculated according to Equation 1.

$$Equation 1$$

$$Quantum {yield}_{Nanoparticles} = Quantum {yield}_{IR 26} \times \frac{{Slope}_{Nanoparticles}}{{Slope}_{IR 26}} \times {( \frac{Refractive index of water}{Refractive index of dichloroethane} )}^{2} ,$$

wherein in Equation 1, the refractive index of water is 1.333, and the refractive index of dichloroethane is 1.4448.

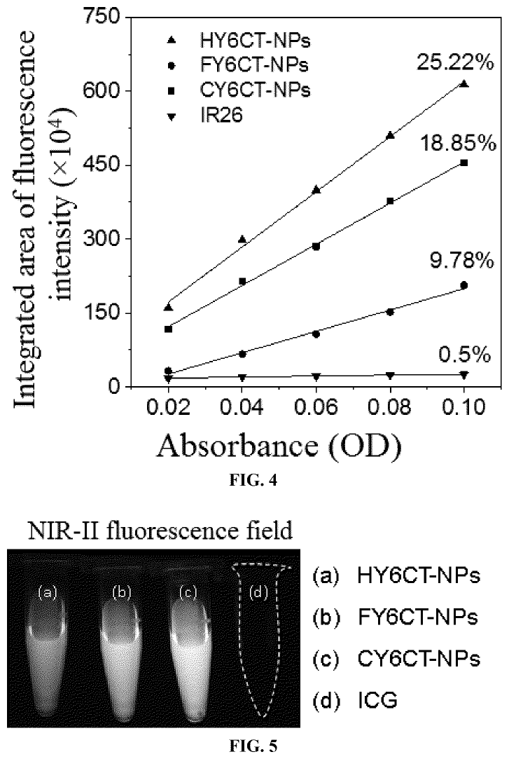

FIG. 4 shows relative quantum yield test results of the nanoimaging agents HY6CT-NPs, FY6CT-NPs and CY6CT-NPs with IR26 as a reference. It can be calculated from FIG. 4 that the relative quantum yields of HY6CT-NPs, FY6CT-NPs and CY6CT-NPs are 25.22%, 18.85% and 9.78%, respectively.

FIG. 5 shows NIR-II fluorescence imagings of aqueous solutions of HY6CT-NPs, FY6CT-NPs, CY6CT-NPs and ICG with the same concentration under excitation of white light (white light illumination). Using NIR-II small animal imager, the PE tubes filled with the same concentration of nano-imaging probes were imaged at a test concentration of 10 μmol·L⁻¹. The results are shown in FIG. 5.

As can be seen from FIG. 5, all three imaging probes could detect obvious NIR-II fluorescence signals under excitation of white light. On the contrary, ICG, as a commercial NIR-II dye, could not detect fluorescence signals under excitation of white light.

### Example 5

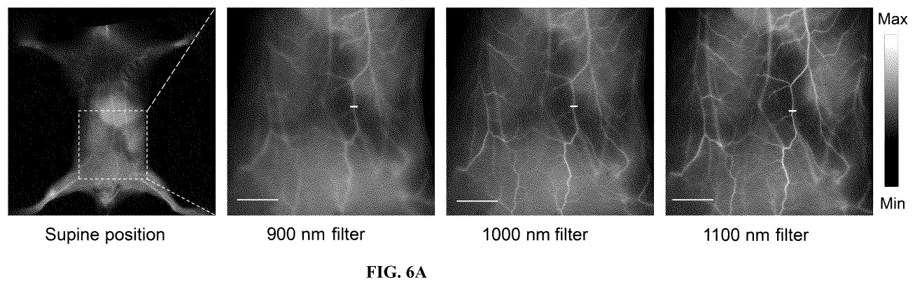

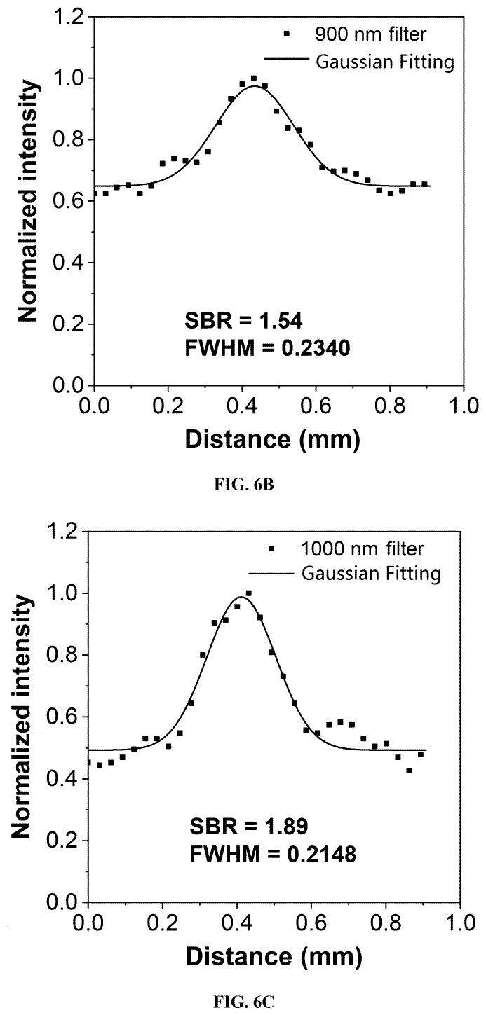

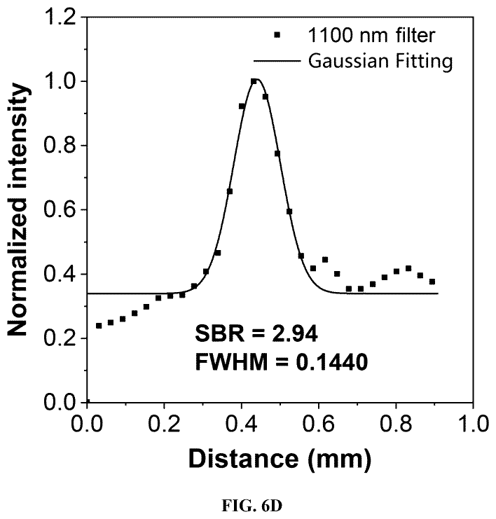

High-resolution NIR-II fluorescence imaging ability test of the fluorescent probe coated nanoparticles FY6CT-NPs on abdominal blood vessels in a mouse: 100 μL of FY6CT-NPs with a concentration of 500 μmol/L was injected into a BALBC/b mouse through a tail vein, and the in vivo fluorescence of the mouse was imaged by an NIR-II small animal in vivo imaging instrument. The excitation light source was the illumination light source (16.5 mW·cm⁻²) of the NIR-II small animal in vivo imaging instrument. Fluorescence images were collected by adjusting filters of different wavelengths. The results are shown in FIG. 6A. The imaging resolutions under different filter conditions were analyzed using an imaging software. The results are shown in FIGS. 6B-6D.

FIG. 6A shows the NIR-II fluorescence imaging of the abdominal blood vessels in a normal mouse with the fluorescent probe coated nanoparticles Y6CT-NPs under different filter conditions. It can be seen from FIG. 6A that after tail-vein injection of FY6CT-NPs, the blood vessels of the mouse are quickly “lighted-up” and fluorescence signals appear in the NIR-II window. As adjusting the filter from 900 nm to 1,100 nm, the clarity of the fluorescence image gradually improves. Under the condition of 1,100 nm filter, abdominal blood vessels are clearly visible and distinguished from surrounding tissues. FIG. 6B-6D show resolution analysis graphs under different filter conditions. As can be seen from FIGS. 6B-6D, as the filter conditions are gradually adjusted, the signal-to-background ratio (SBR) of the imaging gradually increases. Under the condition of 1100 nm filter, SBR reaches 2.94, and the half peak width decreases to 0.1440 mm. These results indicate that FY6CT-NPs have good high-resolution NIR-II fluorescence imaging ability for mouse abdominal blood vessels under excitation of white light.

HY6CT-NPs and CY6CT-NPs are similar to FY6CT-NPs in high-resolution NIR-II fluorescence imaging of mouse abdominal blood vessels under excitation of white light.

From the above examples, it can be seen that, the NIR-II organic nanoimaging agent for vascular imaging under excitation of white light according to the present disclosure has a simple preparation method, shows excellent luminescent properties, and could achieve high-resolution NIR-II imaging of mouse blood vessels under excitation of white light, with good practical effects.

Although the above examples provide a detailed description of the present disclosure, they are only a part, not all of the embodiments. Other embodiments could also be obtained without creative effort based on the present embodiments, all of which shall fall within the scope of the present disclosure.

What is claimed is:

1. A second near-infrared (NIR-II) organic fluorescent probe, having a structure represented by formula I: 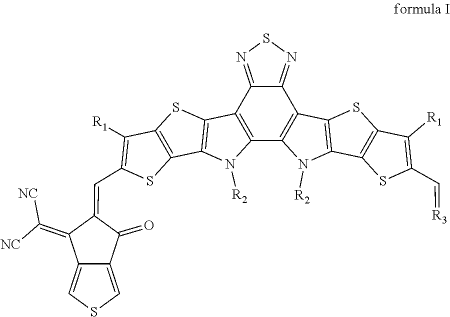
wherein in formula I,
R₁ and R₂ each are independently C₁₋₁₆ branched or linear alkyl; and
R₃ is one selected from the group consisting of 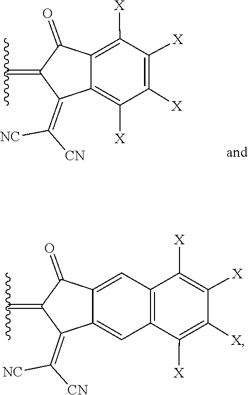
X in R₃ being at least one selected from the group consisting of H and halogen.

2. The NIR-II organic fluorescent probe of claim 1, wherein R₁ and R₂ each are independently C₁₋₁₁ branched or linear alkyl.

3. The NIR-II organic fluorescent probe of claim 1, wherein a plurality of X in R₃ are at least one selected from the group consisting of H, F, Cl and Br.

4. The NIR-II organic fluorescent probe of claim 1, wherein the NIR-II organic fluorescent probe has a structure represented by formula I-a, formula I-b or formula I-c,

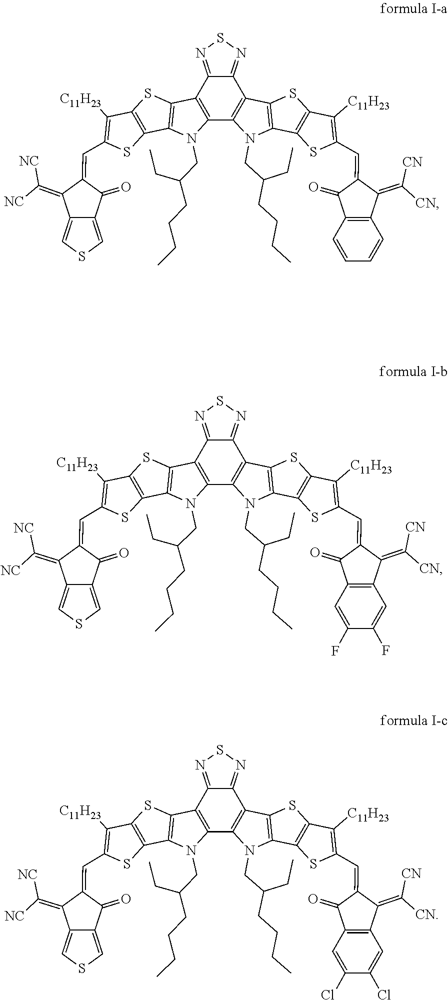

5. A method for preparing the NIR-II organic fluorescent probe of claim 1, comprising the following steps:
mixing a compound having a structure represented by formula II, a compound having a structure represented by formula III, a compound having a structure represented by formula IV or formula V, an organic base catalyst and an organic solvent, and performing a Knoevenagel reaction to obtain the NIR-II organic fluorescent probe having the structure represented by formula I;

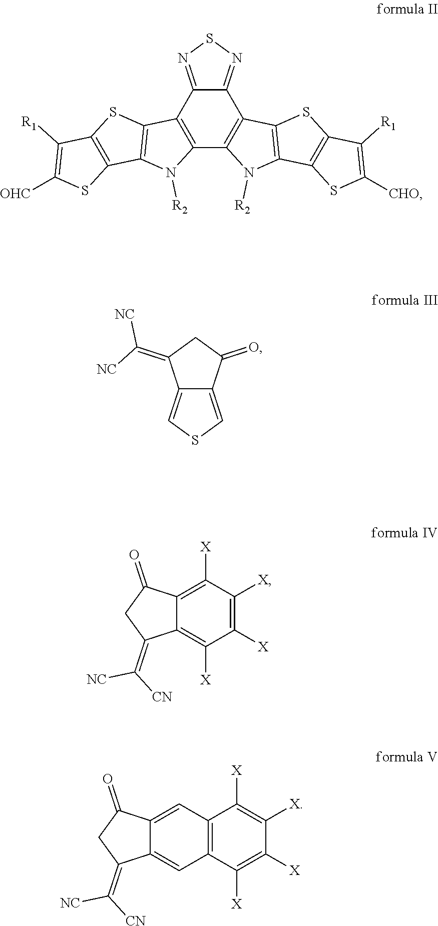

6. The method of claim 5, wherein a molar ratio of the compound having the structure represented by formula II, the compound having the structure represented by formula III, and the compound having the structure represented by formula IV or formula V is 1:1:1,
the organic base catalyst is pyridine, and a molar ratio of the compound having the structure represented by formula II to the organic base catalyst is in a range of 1:3 to 1:10, and
the Knoevenagel reaction is performed at a temperature of 25° C. to 70° C. for 8 h to 24 h.

7. The method of claim 5, wherein the method further comprises after the Knoevenagel reaction, subjecting a resulting Knoevenagel reaction solution to post-treatment, the post-treatment comprising the following steps:
subjecting the resulting Knoevenagel reaction solution to extraction and concentration to obtain a concentrated solution, and
subjecting the concentrated solution to column chromatography separation and recrystallization to obtain a pure product of the NIR-II organic fluorescent probe having the structure represented by formula I,
wherein an eluent used in the column chromatography separation is a mixed solution of dichloromethane and petroleum ether, and a volume ratio of the dichloromethane to the petroleum ether is 2:1; and a solvent used in the recrystallization is a mixture of dichloromethane and n-hexane, and a volume ratio of the dichloromethane to the n-hexane is in a range of 1:3 to 1:6.

8. A fluorescent probe coated nanoparticle, comprising the NIR-II organic fluorescent probe of claim 1, and an organic coating agent coating on a surface of the NIR-II organic fluorescent probe.

9. The fluorescent probe coated nanoparticle of claim 8, wherein the organic coating agent is at least one selected from the group consisting of methoxypolyethylene glycol amine, 1,2-distearoyl-sn-glycero-3-phosphoethanolamine-poly(ethyleneglycol), 1,2-distearoyl-sn-glycero-3-phosphoethanolamine-N-[maleimide(polyethylene glycol)], 1,2-distearoyl-sn-glycero-3-phosphoethanolamine-N-[folate(polyethylene glycol)], 1,2-distearoyl-sn-glycero-3-phosphoethanolamine-N-[thiol(polyethylene glycol)], 1,2-distearoyl-sn-glycero-3-phosphoethanolamine-N-[carboxy(polyethylene glycol)], 1,2-distearoyl-sn-glycero-3-phosphoethanolamine-N-[azido(polyethylene glycol)], 1,2-distearoyl-sn-glycero-3-ethanolamine-N-[biotin(polyethylene glycol)], 1-palmitoyl-2-oleoyl-sn-glycero-3-phosphoethanolamine, 1-stearoyl-2-oleoyl lecithin, 1,2-dipalmitoyl phosphoethanolamine-polyethylene glycol, polystyrene-grafted-poly(ethylene glycol), methoxy PEG polylactic acid-hydroxyacetic acid copolymer, and Pluronic® F-127.

10. The fluorescent probe coated nanoparticle of claim 8, wherein the fluorescent probe coated nanoparticle has a particle size of 50 nm to 200 nm.

11. A method for preparing the fluorescent probe coated nanoparticle of claim 8, comprising the following steps:
mixing the NIR-II organic fluorescent probe,, an organic coating agent and an organic solvent to obtain a mixed solution; and
mixing the mixed solution with water, and performing ultrasonic coprecipitation to obtain the fluorescent probe coated nanoparticle.

12. The method of claim 11, wherein the ultrasonic coprecipitation is performed at a powder of 100 W to 200 W for 3 min to 10 min.

13. A bioimaging contrast agent, comprising the NIR-II organic fluorescent probe of claim 1.

14. The NIR-II organic fluorescent probe of claim 2, wherein the NIR-II organic fluorescent probe has a structure represented by formula I-a, formula I-b or formula I-c,

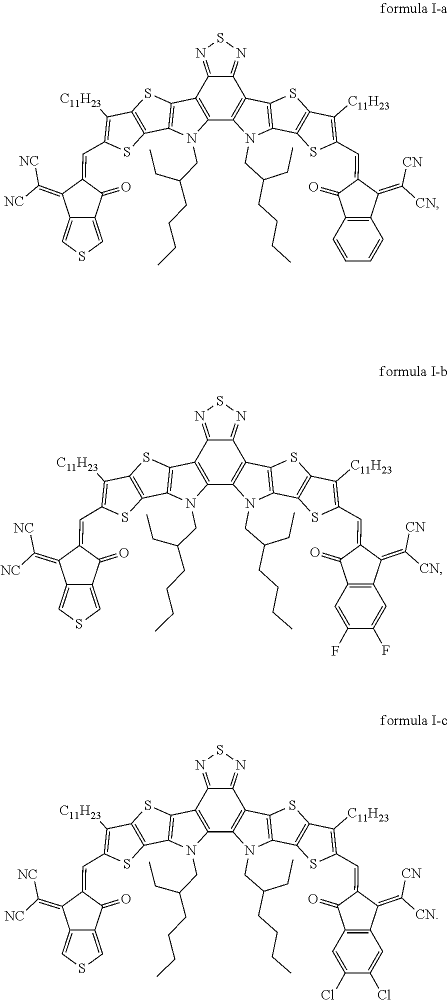

15. The NIR-II organic fluorescent probe of claim 3, wherein the NIR-II organic fluorescent probe has a structure represented by formula I-a, formula I-b or formula I-c,

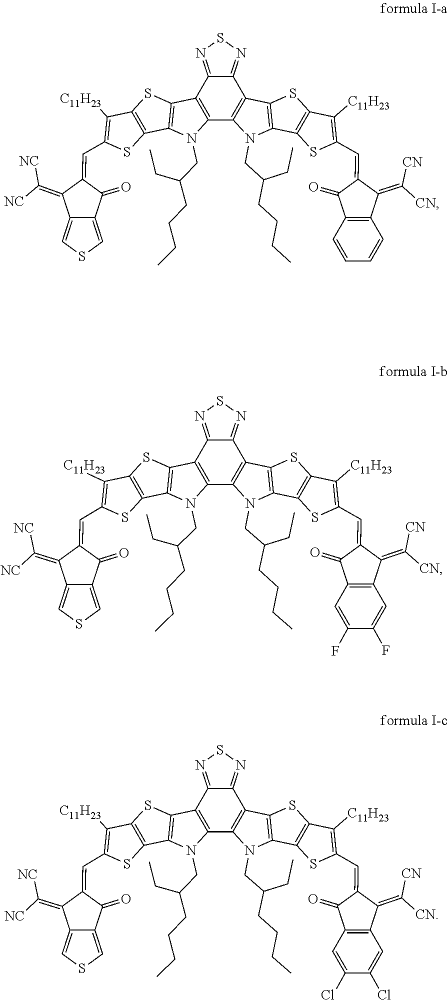

16. The method of claim 5, wherein R₁ and R₂ each are independently C₁₋₁₁ branched or linear alkyl.

17. The method of claim 5, wherein a plurality of X in R₃ are at least one selected from the group consisting of H, F, Cl and Br.

18. The method of claim 5, wherein the NIR-II organic fluorescent probe has a structure represented by formula I-a, formula I-b or formula I-c,

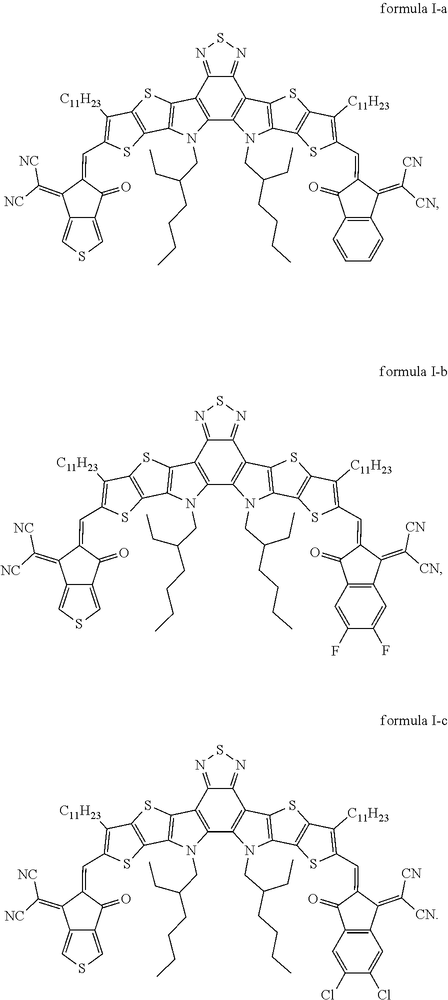

19. The fluorescent probe coated nanoparticle of claim 8, wherein the NIR-II organic fluorescent probe is prepared by a method comprising the following steps:
mixing a compound having a structure represented by formula II, a compound having a structure represented by formula III, a compound having a structure represented by formula IV or formula V, an organic base catalyst and an organic solvent, and performing a Knoevenagel reaction to obtain the NIR-II organic fluorescent probe having the structure represented by formula I;

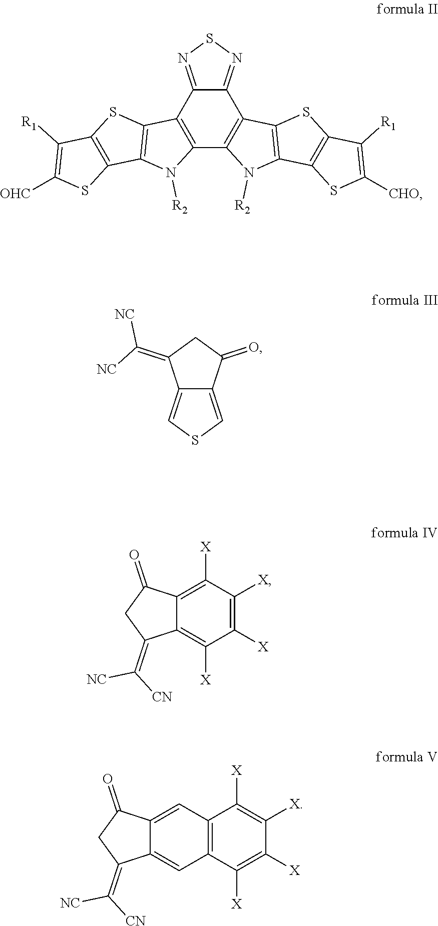

20. A bioimaging contrast agent, comprising the fluorescent probe coated nanoparticle of claim 8.
